(19) United States
(12) Patent Application Publication
(10) Pub. No.: US 2026/0253027 A1
(43) Pub. Date: Aug. 27, 2026

(54) AUTOMATIC ODOR DETECTION AND MITIGATION SYSTEM FOR INSPECTION OF INCOMING TRUCK TRAILER

(71) Applicant: NIAGARA BOTTLING, LLC, Diamond Bar, CA (US)

(72) Inventors: Nuri Jonathan Dimler, Laguna Beach, CA (US); William Hall, La Habra Heights, CA (US); Andrew Dimitri Peykoff, Sr., Newport Coast, CA (US); Sudheer Bandla, Rancho Cucamonga, CA (US); Huong Khuat Ngo, Garden Grove, CA (US); Luis Alberto Ramos Vittini, Los Angeles, CA (US); Robert Dale Leathers, Redlands, CA (US)

(21) Appl. No.: 19/125,637

(22) PCT Filed: Nov. 10, 2023
(86) PCT No.: PCT/US2023/079379

§ 371 Date: Apr. 29, 2025

Related U.S. Application Data
(60) Provisional application No. 63/383,350, filed on Nov. 11, 2022.

(51) Int. Cl. G06Q 10/083; A61L 9/14; G01N 33/00

(52) U.S. Cl. CPC G06Q 10/0841; A61L 9/145; G01N 33/0027; A61L 2209/111

(57) ABSTRACT

Devices, methods, and non-transitory computer-readable media for automatic odor detection and mitigation of incoming truck trailer. In one example, a controller may include a memory and an electronic processor communicatively coupled to the memory. The electronic processor is configured to receive, from an odor sensor, a sensor signal indicative of an odor sensor output, analyze an odor profile of a truck trailer based on the odor sensor output, determine a response based at least in part on the odor profile, and generate a control signal based on the response.

### CROSS-REFERENCE TO RELATED APPLICATION

This application claims priority to, and the benefit of, U.S. Provision Application No. 63/383,350, filed on Nov. 11, 2022, the entire contents of which is incorporated herein by reference.

### FIELD The present invention relates to systems and methods for inspecting a truck trailer prior to loading.

### BACKGROUND

Incoming truck trailers arrive at a facility for unloading of cargo transported from another site and/or loading of new cargo to be transported to another site. A trailer may be inspected prior to loading the trailer with new cargo.

### SUMMARY

Examples of some characteristics that may be evaluated during this pre-loading inspection include visible damage, the presence of unloaded cargo in the trailer, and odors within the trailer. If unfavorable and/or potentially dangerous odors are detected, the trailer may be rejected and the new cargo loaded onto a different trailer instead. In some implementations, additional variables are also measured and considered such as, for example, temperature, humidity, concentration of a detected odor, and toxicity of the detected odor. Because odor is generally a subjective experience, a trailer that is determined to be acceptable by one person may be rejected by another person. Additionally, factors such as deadlines, stress, and illness may affect a human's sense of smell and cause an employee to incorrectly accept and load a trailer that exhibits an undesirable odor, which could lead to damaged cargo and/or unhappy customers.

In some aspects, the present disclosure described herein relate to a controller including: a memory; and an electronic processor communicatively coupled to the memory, the electronic processor configured to receive, from an odor sensor, a sensor signal indicative of an odor sensor output, analyze an odor profile of a truck trailer based on the odor sensor output, determine a response based at least in part on the odor profile, and generate a control signal based on the response.

In some aspects, the present disclosure described herein relate to a method including: receiving, with a controller, a sensor signal indicative of an odor sensor output from an odor sensor; analyzing, with the controller, an odor profile of a truck trailer based on the odor sensor output; determining, with the controller, a response based at least in part on the odor profile; and generating, with the controller, a control signal based on the response.

In some aspects, the present disclosure described herein relate to a non-transitory computer-readable medium including instructions that, when executed by an electronic processor, cause the electronic processor to perform a set of operations including: receiving a sensor signal indicative of an odor sensor output from an odor sensor; analyzing an odor profile of a truck trailer based on the odor sensor output; determining a response based at least in part on the odor profile; and generating a control signal based on the response.

In some implementations, the present disclosure provides an automatic odor detection and mitigation system configured to perform a pre-loading inspection of a trailer. When a trailer arrives at a facility to be loaded with new cargo (or, in some implementations, before the trailer arrives at the facility), the automatic odor detection and mitigation system collects data on odors and environmental conditions within the trailer including, for example, names of chemicals detected by an odor sensor, strength/concentration of the detected odors, temperature within the trailer, and humidity within the trailer. The system then evaluates the collected data using an algorithm to determine if the truck trailer should be accepted or rejected. The system then automatically initiates the process of accepting or rejecting the trailer/truck.

In some implementations, the system initiates the rejection of a trailer/truck by activating a warning output device or transmitting a warning signal to another computer system. In some implementations, the system initiates the process of accepting or rejecting the trailer/truck by transmitting a signal to a communication device within the operator cab of the truck pulling the trailer, which in turn outputs an indication to an operator of the truck indicative of whether the trailer has been accepted or rejected. In some implementations, the system initiates the process of accepting or rejecting the trailer/truck by transmitting an output signal indicative of the acceptance/rejection to a truck/trailer management logistics system.

Additionally, in some implementations, the system is configured to further determine whether a detected odor can be mitigated and, based on the determination, automatically performs an appropriate mitigation. For example, in some implementations, the system further includes one or more sprayers and, in response to detecting an odor in the trailer, the system automatically operates the sprayer to emit a sanitizing or odor neutralizing agent into the trailer. In some implementations, the system is equipped with multiple sprayers each configured to emit a different type of sanitizing or odor neutralizing agent and the system is further configured to determine which sanitizing or odor neutralizing agent is appropriate for mitigating an odor profile detected in the trailer and operates the sprayer to emit the selected appropriate sanitizing or odor neutralizing agent.

In some implementations, the system includes one or more sensors (e.g., an odor sensor) installed within a specific trailer. In other implementations, the one or more sensors are installed at a location external to the trailer where the pre-loading inspections are performed (e.g., at a loading dock of the facility). In other implementations, the system utilizes one or more sensors incorporated into a handheld device that is carried by a user into the trailer for the pre-loading inspection. In still other implementations, the system includes an autonomous, semi-autonomous, or remotely operated vehicle that is equipped with one or more sensors for performing the pre-loading inspection and the vehicle operated to travel into the trailer to perform the pre-loading inspection.

In some implementations, the system may include one or more databases storing data characteristics of odors that are commonly found in trailers defined, for example, by temperature, humidity, concentration, and/or strength of the odor. In some implementations, the system includes or is configured to receive data from one or more sensors or instruments including, for example, sensors configured to detect odors and/or environmental conditions in the trailer. In some implementations, the system includes software instructions stored on a non-transitory computer-readable memory that, when executed by an electronic processor, cause the system to collect data from the sensors and to automatically determine whether to accept or rejecting the trailer/truck and, in some implementations, to perform one or more mitigation operation based on a detected odor profile for the trailer. Finally, in some implementations where the odor detection and mitigation system is integrated into an autonomous, semi-autonomous, or remotely operated vehicle, the system may also include software and hardware that is further configured to cause the vehicle to move into the trailer, initiate the odor detection/analysis routine, and then automatically accept/reject the trailer and/or perform the mitigation operation.

Other aspects of the present disclosure will become apparent by consideration of the detailed description and accompanying drawings.

### BRIEF DESCRIPTION OF THE DRAWINGS

FIG. 1 is a block diagram of a control system for an odor detection and mitigation system for conducting pre-loading trailer inspections according to one embodiment.

FIG. 2 is a flowchart of a method performed by the system of FIG. 1 for conducting a pre-loading trailer inspection.

FIG. 3 is a flowchart of another example of a method performed by the system of FIG. 1 for conducting a pre-loading trailer inspection.

FIG. 4 is a schematic diagram of an example of a machine-learning model used by the system of FIG. 1 to analyze odors and/or identify appropriate mitigation operations according to some embodiments.

### DETAILED DESCRIPTION

Before any embodiments of the present disclosure are explained in detail, it is to be understood that the present disclosure is not limited in its application to the details of construction and the arrangement of components set forth in the following description or illustrated in the following drawings. The present disclosure is capable of other embodiments and of being practiced or of being carried out in various ways.

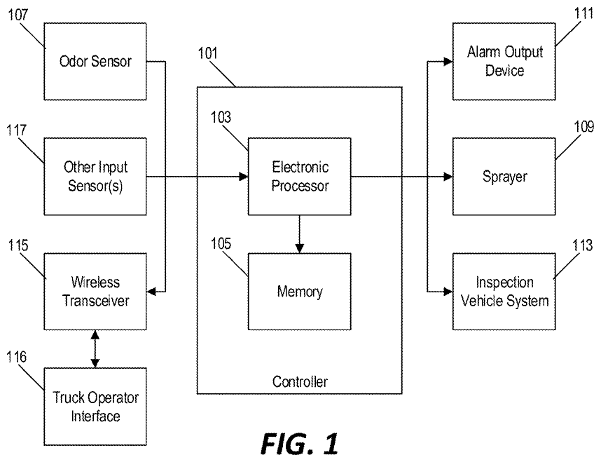

FIG. 1 illustrates an example of an odor detection and mitigation system for conducting pre-loading inspection of trailers. The system includes a controller 101 with an electronic processor 103 and a non-transitory computer-readable memory 105. The memory 105 is communicatively coupled to the electronic processor 103 and stores data and computer-executable instructions that, when executed by the electronic processor 103 provides the functionality of the controller 101 (including, for example, the functionality described in the examples herein).

The controller 101 is communicatively coupled to an odor sensor 107 and is configured to receive one or more output signals from the odor sensor 107. In some implementations, the odor sensor 107 includes a plurality of sensors (e.g., biosensors and silicon photonics) and, in some such implementations, machine-learning algorithms are applied to the output of the sensors to detect and classify odors. In other implementations, the odor sensor 107 is configured to detect specific chemical compounds in the air that are responsible for undesirable odors and, in some such implementations, the odor sensor 107 includes one or more sensors configured to detect & quantify those specific chemical compounds and to produce an output signal indicative of a relative amount of the detected chemical compounds (e.g., indicative of a “strength” of an odor corresponding to a particular compound). In some implementations, the odor sensor 107 includes instrumentation configured to detect specific gases or volatile organic compounds (VOCs) including, for example, gases that are known to be flammable, combustible, or toxic. These gases may or may not be associated with “bad” (e.g., unpleasant) odors, but can have other negative effects on cargo if the cargo is loaded into a trailer while the gases are present. In some implementations, the odor sensor 107 may be olfaction device also referred to as an “electronic nose” device.

In the example of FIG. 1, the controller 101 is communicatively coupled to a sprayer 109. The sprayer 109 includes or is coupled to a reservoir storing a sanitizing or odor neutralizing agent and the sprayer 109 is configured to controllably and selectively emit the agent (e. g, in liquid, gas, or aerosolized form) in response to a control signal received by the sprayer 109 from the controller 101. In some implementations, the system may include multiple different sprayers 109 each configured to emit a different type of sanitizing or odor neutralizing agent. In some implementations, a single sprayer 109 may be coupled to multiple different reservoirs each storing a different type of sanitizing or odor neutralizing agent and the sprayer 109 is configured to selectively emit different types of agents by selectively emitting the agent from a particular reservoir of the multiple reservoirs in response to the control signal received from the controller 101.

In the example of FIG. 1, the controller 101 is also communicatively coupled to an alarm output device such as, for example, a speaker or a light-based device configured to generate an auditory and/or visual warning output in response to a signal received from the controller 101. Accordingly, as discussed in further detail below, the controller 101 is configured to analyze the output data received from the odor sensor 107 and evaluate an odor profile for the trailer as a part of the pre-loading inspection. If the system detects odor that can be effectively mitigation by use of a sanitizing or odor neutralizing agent, then the controller 101 causes the sprayer 109 to emit the appropriate sanitizing or odor neutralizing agent. In some implementations, the controller 101 may also be configured to operate the alarm output device 111 to generate the warning output in addition to operating the sprayer 109 in response to detecting an odor that can be effectively mitigated. And, in some implementations, the controller 101 is configured to operate the alarm output device 111 to generate a warning output in response to detecting an odor that cannot be effectively mitigated. In some implementations, the controller 101 is configured to operate the alarm output device 111 to generate different types of warning outputs depending on whether the detected odor is one that can be effective mitigated or is one that cannot be effectively mitigated. In some implementations, a warning output indicative of a detected odor that cannot be effectively mitigated indicates that the trailer under inspection should be rejected and that new cargo should not be loaded into the trailer.

In some implementations, the system of FIG. 1 is installed at a fixed location either near a loading dock at the loading facility or within the trailer itself. In other implementations, the system of FIG. 1 may include a handheld device equipped with the odor sensor 107 that is carried by a user into the trailer to perform the pre-loading inspection. In still other implementations, the odor sensor 107 is installed on or incorporated into an autonomous, semi-autonomous, or remotely-operated vehicle configured to perform the pre-loading inspections. As illustrated in the example of FIG. 1, in some such implementations, the controller 101 is communicatively coupled to the inspection vehicle system 113 and is configured to communicate with the inspection vehicle (i.e., the vehicle conducting the inspection) during the pre-loading inspection. In some implementations, the controller 101 may be configured to generate and transmit control signals to the inspection vehicle system 113 to control the operation of the inspection vehicle during the pre-loading inspection. For example, in some implementations, the controller 101 may be configured to cause the inspection vehicle to move into the trailer when a new truck arrives at the facility, to operate the odor sensor 107 to collect data from the trailer, to operate the sprayer 109 and/or alarm output device 111 based on an analysis of the data from the odor sensor 107, and to cause the inspection vehicle to move out of the trailer after completing the pre-loading inspection.

In the example of FIG. 1, the controller 101 is also communicatively coupled to a wireless transceiver 115 that allows the controller 101 to communicate with other remote computing systems and devices including, for example, a truck/trailer management logistics system. In some implementations, the controller 101 is configured to communicate through the wireless transceiver 115 with one or more computing system of the truck that is pulling the trailer under inspection such as, for example a truck operator user interface 116 positioned within the operator cab of the truck. For example, in some implementations, the controller 101 may be configured to receive information from the truck indicative of a prior cargo, etc. and/or may be configured to send information to the truck after completion of the pre-loading inspection indicating to the truck (or, e.g., an operator of the truck) whether the trailer has been accepted or rejected. For example, in implementations where the odor sensor 107 is installed within the interior of the trailer, the system may be configured to perform the odor-based pre-loading inspection before the trailer arrives at the loading facility and, in the event that the trailer is rejected based on the inspection, the controller 101 transmits a signal to the truck operator user interface device 116 located within the cab of the truck pulling the trailer to inform the operator that the trailer has been rejected and/or instructing the operator to transport the trailer to a different location.

Finally, in some implementations, the controller 101 may also be communicatively coupled to one or more other input sensors 117. For example, in some implementations, the controller 101 is configured to receive data from one or more environmental sensors that are configured to detect and convey to the controller 101 information regarding the environment inside the trailer such as, for example, temperature and/or humidity. Additionally or alternatively, in some implementations, the one or more other input sensors 117 may include a camera and/or a laser scanning system and the controller 101 may be configured to automatically detect, based on captured visual data, unloaded cargo in the trailer and/or certain types of physical damage to the interior of the trailer.

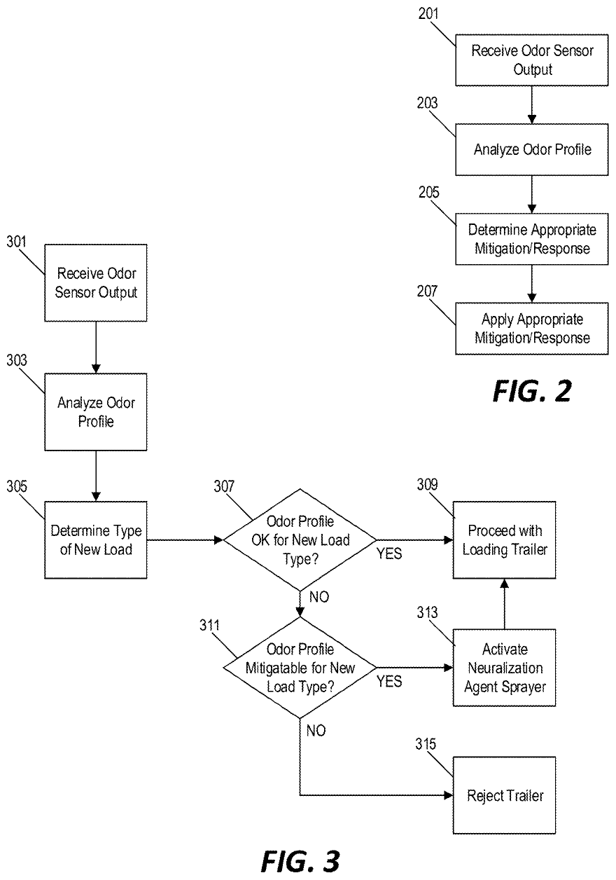

As illustrated in the example of FIG. 2, the controller 101 is configured to receive the output from the odor sensor 107 (step 201), to analyze an odor profile for the trailer based on the output of the odor sensor 107 (step 203), to determine an appropriate mitigation or action in response to the odor profile for the trailer (step 205), and to apply that mitigation or other action (step 207). In some implementations, as discussed above, the appropriate mitigation for some detected odor profiles may include operating a sprayer 109 to emit a particular sanitizing or odor neutralizing agent into the trailer and, in some implementations, the system may be configured to selectively emit one or several different types of agents. Additionally or alternatively, in some implementations, the appropriate action for a particular detected odor profile may include operating the alarm output device 111 or either accepting or rejecting the trailer based on the detected odor profile.

FIG. 3 illustrates a more detailed example of the method of FIG. 2. In this example, in additional to receive the odor sensor output (step 301) and analyzing the odor profile for the trailer (step 303), the controller 101 is also configured to determine the new cargo to be loaded onto the trailer (step 305). As some odors (and their associated chemical compounds and gases) may affect different types of cargos differently, the appropriate mitigation for a given odor profile may be different depending on the new type of cargo to be loaded onto the trailer. For example, for some cargos the application of an odor neutralizing agent may be sufficient mitigation for a particular type of odor, but, for a different type of cargo, detection of the same odor may require that the trailer be rejected.

After analyzing the odor profile for the trailer (step 303) and determining the type of cargo for the new load (step 305), the controller 101 determines whether the odor profile of the trailer is acceptable for the new load type (step 307). If so, then the system automatically initiates loading of the trailer (e.g., automated loading, semi-automated loading, or manual loading) without any mitigation (step 309). However, if the detected odor profile is not acceptable for the new load (step 307), then the controller 101 determines whether the odor profile can be effectively mitigated (step 311). If the controller 101 determines that the detected odor profile can be effectively mitigated by the system (e.g., by use of a sanitizing or odor neutralizing agent), then the controller 101 causes the sprayer 109 to emit the appropriate agent (step 313) before initiating the loading of the trailer (step 309). Finally, if the controller 101 determines that the detected odor profile for the trailer is not acceptable for the new load type and cannot be effectively mitigated, then the controller 101 activates the alarm output device 111 and initiates a rejection of the trailer (step 315). In some implementations, this rejection of the trailer is automatically communicated to an operator of the truck pulling the trailer and instructs the operator to remove the rejected trailer from the facility.

In some implementations, the odor profile includes an identification of one or more specific chemical compounds (e.g., gases) detected by the odor sensor. The specific chemical compound may be determined by the odor sensor 107 itself and communicated to the controller 101 or, in some implementations, the controller 101 is configured to determine an identity of the one or more specific chemical compound by analyzing the output data received from the odor sensor 107. In other implementations, the odor profile may include quantified values for a plurality of different variables measured by the odor sensor 107 that, although indicative of certain odors, might not explicitly identify the odor.

In some implementations, the memory 105 stores one or more databases storing a list of appropriate mitigation techniques, automated acceptance/rejection conditions, and/or other information corresponding to different odor profiles. In some implementations, the memory 105 may be configured to store a look-up table that identifies an appropriate action for each odor profile, range of odor profiles, and/or combination of odor profile and cargo type. Accordingly, for a given odor profile (and, in some cases, a give combination of odor profile and cargo type), the look-up table will indicate (1) that the trailer should be rejected, (2) that the trailer should be accepted without mitigation, or (3) a particular mitigation that should be applied to the trailer before the trailer is loaded.

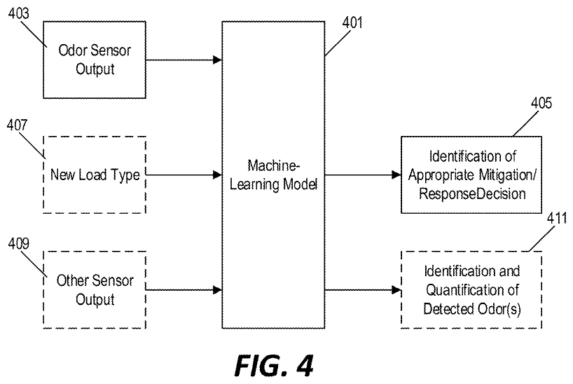

In some implantations, the system is configured to utilize one or more trained machine-learning models in addition to or instead of the databases/look-up tables. FIG. 4 illustrates an example of one such trained machine-learning model. In the example of FIG. 4, the machine-learning model 401 is configured to receive as input the output data 403 collected from the odor sensor 107. In response to receiving the collected odor sensor data 403, the trained machine-learning model 401 produces an output 405 identifying an appropriate mitigation/response (e.g., accept trailer, reject trailer, or apply a mitigation action). Accordingly, in some implementations, the method of FIG. 2 is performed by the controller 101 by receiving the odor sensor output (step 201), applying the machine-learning model to the odor sensor data (steps 203 & 205), and then operating the system in accordance with the output of the machine-learning model (step 207).

In some implementations, the machine-learning model may be trained to receive other inputs in addition to or instead of the odor sensor output data 403. For example, as illustrated in the example of FIG. 4, the machine-learning model 401 in some implementations is trained to receive as input an identification of the new load type 407 (i.e., the type of cargo that will be loaded onto the trailer if it passes the inspection) and/or other sensor output data 409 (e.g., temperature, humidity, camera image data, optical scan data, etc.).

As discussed above, in some implementations, the machine-learning model 401 is trained to produce as output an identification 405 of a particular mitigation action and/or a determination of whether the trailer should be accepted or rejected. However, in other implementations, the machine-learning model 401 may be trained to produce as output an identification 411 (and, in some implementations, a quantification of concentration, strength, etc.) of specific odors detected in the trailer. In some implementations, the machine-learning model 401 is trained to produce as output both the identification of the appropriate mitigation/response 405 and the identification of the detected odor 411. However, in some other implementations, the machine-learning model 401 may instead be configured to produce as output the identification/quantification of the detected odors 411, but not to produce as output the identification of an appropriate mitigation/response 405. In some such implementations, the controller 101 may be configured to then use another mechanism such as an algorithm, database, and/or look-up table to determine an appropriate mitigation/response for the trailer based on the identification/quantification of the odors 411 provided as output by the machine-learning model 401.

One example method described herein includes receiving, with an electronic processor, an output of an odor sensor, the output indicative of an odor profile of a trailer; analyzing, with the electronic processor, the output; determining, with the electronic processor, a response based on the analysis of the output; and applying, with the electronic processor, the response to the trailer.

The following are enumerated examples of devices, methods, and non-transitory computer-readable media of the present disclosure. Example 1: a controller comprising: a memory; and an electronic processor communicatively coupled to the memory, the electronic processor configured to receive, from an odor sensor, a sensor signal indicative of an odor sensor output, analyze an odor profile of a truck trailer based on the odor sensor output, determine a response based at least in part on the odor profile, and generate a control signal based on the response.

Example 2: the controller of Example 1, wherein, to determine the response based at least in part on the odor profile, the electronic processor is further configured to determine a type of load of the truck trailer, and determine whether characteristics of the odor profile are within acceptable ranges for the type of load.

Example 3: the controller of Example 2, wherein, in response to determining that characteristics of the odor profile are within the acceptable ranges for the type of load, the control signal represents an instruction to proceed with loading the truck trailer.

Example 4: the controller of Examples 2 or 3, wherein, to determine the response based at least in part on the odor profile, the electronic processor is further configured to determine whether the odor profile is mitigatable for the type of load.

Example 5: the controller of Example 4, wherein, in response to determining that the odor profile is mitigatable for the type of load, the control signal represents an instruction to a sprayer system to spray a neutralization agent to mitigate one or more odors.

Example 6: the controller of Example 4, wherein in response to determining that the odor profile is not mitigatable for the type of load, the control signal represents an instruction to a docking bay to reject the truck trailer.

Example 7: the controller of Examples 1-6, wherein the memory includes a machine learning model, and wherein the electronic processor is further configured to determine, with the machine learning model, the response based at least in part on the odor profile.

Example 8: the controller of Examples 1-7, wherein the electronic processor is further configured to output the control signal to one or more devices from a group consisting of: an alarm output device, a sprayer, and an inspection vehicle system.

Example 9: a method comprising: receiving, with a controller, a sensor signal indicative of an odor sensor output from an odor sensor; analyzing, with the controller, an odor profile of a truck trailer based on the odor sensor output; determining, with the controller, a response based at least in part on the odor profile; and generating, with the controller, a control signal based on the response.

Example 10: the method of Example 9, wherein determining the response based at least in part on the odor profile further includes determining a type of load of the truck trailer, and determining whether characteristics of the odor profile are within acceptable ranges for the type of load.

Example 11: the method of Example 10, wherein the control signal represents an instruction to proceed with loading the truck trailer in response to determining that the odor profile is within acceptable ranges for the type of load.

Example 12: the method of Examples 10 or 11, wherein determining the response based at least in part on the odor profile further includes determining whether the odor profile is mitigatable for the type of load.

Example 13: the method of Example 12, wherein the control signal represents an instruction to a sprayer system to spray a neutralization agent to mitigate one or more odors in response to determining that the odor profile is mitigatable for the type of load.

Example 14: the method of Example 12, wherein the control signal represents an instruction to a docking bay to reject the truck trailer in response to determining that the odor profile is not mitigatable for the type of load.

Example 15: the method of Examples 9-14, wherein determining, with the controller, the response based at least in part on the odor profile further includes determining, with the controller and a machine learning model, the response based at least in part on the odor profile.

Example 16: the method of Examples 9-15, further comprising: outputting the control signal to one or more devices from a group consisting of: an alarm output device, a sprayer, and an inspection vehicle system.

Example 17: a non-transitory computer-readable medium comprising instructions that, when executed by an electronic processor, cause the electronic processor to perform a set of operations comprising: receiving a sensor signal indicative of an odor sensor output from an odor sensor; analyzing an odor profile of a truck trailer based on the odor sensor output; determining a response based at least in part on the odor profile; and generating a control signal based on the response.

Example 18: the non-transitory computer-readable medium of Example 17, wherein determining the response based at least in part on the odor profile further includes determining a type of load of the truck trailer, and determining whether characteristics of the odor profile are within acceptable ranges for the type of load.

Example 19: the non-transitory computer-readable medium of Example 18, wherein the control signal represents an instruction to proceed with loading the truck trailer in response to determining that the odor profile is within acceptable ranges for the type of load.

Example 20: the non-transitory computer-readable medium of Example 18, wherein determining the response based at least in part on the odor profile further includes determining whether the odor profile is mitigatable for the type of load, wherein the control signal represents an instruction to a sprayer system to spray a neutralization agent to mitigate one or more odors in response to determining that the odor profile is mitigatable for the type of load, and wherein the control signal represents an instruction to a docking bay to reject the truck trailer in response to determining that the odor profile is not mitigatable for the type of load.

Accordingly, the devices, methods, and non-transitory computer-readable media described herein provide, among other things, a controller for automatically performing a pre-loading odor-based inspection of a trailer and operating various systems and devices in response to the outcome of the inspection.

What is claimed is:

1. A controller comprising:
a memory; and
an electronic processor communicatively coupled to the memory, the electronic processor configured to
receive, from an odor sensor, a sensor signal indicative of an odor sensor output,
analyze an odor profile of a truck trailer based on the odor sensor output,
determine a response based at least in part on the odor profile, and
generate a control signal based on the response.

2. The controller of claim 1, wherein, to determine the response based at least in part on the odor profile, the electronic processor is further configured to
determine a type of load of the truck trailer, and
determine whether characteristics of the odor profile are within acceptable ranges for the type of load.

3. The controller of claim 2, wherein, in response to determining that characteristics of the odor profile are within the acceptable ranges for the type of load, the control signal represents an instruction to proceed with loading the truck trailer.

4. The controller of claim 2, wherein, to determine the response based at least in part on the odor profile, the electronic processor is further configured to determine whether the odor profile is mitigatable for the type of load.

5. The controller of claim 4, wherein, in response to determining that the odor profile is mitigatable for the type of load, the control signal represents an instruction to a sprayer system to spray a neutralization agent to mitigate one or more odors.

6. The controller of claim 4, wherein in response to determining that the odor profile is not mitigatable for the type of load, the control signal represents an instruction to a docking bay to reject the truck trailer.

7. The controller of claim 1, wherein the memory includes a machine learning model, and wherein the electronic processor is further configured to determine, with the machine learning model, the response based at least in part on the odor profile.

8. The controller of claim 1, wherein the electronic processor is further configured to output the control signal to one or more devices from a group consisting of:
an alarm output device,
a sprayer, and
an inspection vehicle system.

9. A method comprising:
receiving, with a controller, a sensor signal indicative of an odor sensor output from an odor sensor;
analyzing, with the controller, an odor profile of a truck trailer based on the odor sensor output;
determining, with the controller, a response based at least in part on the odor profile; and
generating, with the controller, a control signal based on the response.

10. The method of claim 9, wherein determining the response based at least in part on the odor profile further includes
determining a type of load of the truck trailer, and
determining whether characteristics of the odor profile are within acceptable ranges for the type of load.

11. The method of claim 10, wherein the control signal represents an instruction to proceed with loading the truck trailer in response to determining that the odor profile is within acceptable ranges for the type of load.

12. The method of claim 10, wherein determining the response based at least in part on the odor profile further includes determining whether the odor profile is mitigatable for the type of load.

13. The method of claim 12, wherein the control signal represents an instruction to a sprayer system to spray a neutralization agent to mitigate one or more odors in response to determining that the odor profile is mitigatable for the type of load.

14. The method of claim 12, wherein the control signal represents an instruction to a docking bay to reject the truck trailer in response to determining that the odor profile is not mitigatable for the type of load.

15. The method of claim 9, wherein determining, with the controller, the response based at least in part on the odor profile further includes determining, with the controller and a machine learning model, the response based at least in part on the odor profile.

16. The method of claim 9, further comprising:
outputting the control signal to one or more devices from a group consisting of:
an alarm output device,
a sprayer, and
an inspection vehicle system.

17. A non-transitory computer-readable medium comprising instructions that, when executed by an electronic processor, cause the electronic processor to perform a set of operations comprising:
receiving a sensor signal indicative of an odor sensor output from an odor sensor;
analyzing an odor profile of a truck trailer based on the odor sensor output;
determining a response based at least in part on the odor profile; and
generating a control signal based on the response.

18. The non-transitory computer-readable medium of claim 17, wherein determining the response based at least in part on the odor profile further includes
determining a type of load of the truck trailer, and
determining whether characteristics of the odor profile are within acceptable ranges for the type of load.

19. The non-transitory computer-readable medium of claim 18, wherein the control signal represents an instruction to proceed with loading the truck trailer in response to determining that the odor profile is within acceptable ranges for the type of load.

20. The non-transitory computer-readable medium of claim 18, wherein determining the response based at least in part on the odor profile further includes determining whether the odor profile is mitigatable for the type of load,
wherein the control signal represents an instruction to a sprayer system to spray a neutralization agent to mitigate one or more odors in response to determining that the odor profile is mitigatable for the type of load, and
wherein the control signal represents an instruction to a docking bay to reject the truck trailer in response to determining that the odor profile is not mitigatable for the type of load.
